(19) United States
(12) Patent Application Publication
(10) Pub. No.: US 2019/0167116 A1
(43) Pub. Date: Jun. 6, 2019

(54) OPTICAL REDOX IMAGING SYSTEMS AND METHODS

(71) Applicants: Yu Chen, College Park, MD (US); Qinggong Tang, College Park, MD (US); Udayakumar Kanniyappan, Hyattsville, MD (US); Lin Z. Li, Aston, PA (US); He Xu, Lansdowne, PA (US); Yi Liu, College Park, MD (US); Emily Conant, Philadelphia, PA (US)

(72) Inventors: Yu Chen, College Park, MD (US); Qinggong Tang, College Park, MD (US); Udayakumar Kanniyappan, Hyattsville, MD (US); Lin Z. Li, Aston, PA (US); He Xu, Lansdowne, PA (US); Yi Liu, College Park, MD (US); Emily Conant, Philadelphia, PA (US)

(21) Appl. No.: 16/192,229

(22) Filed: Nov. 15, 2018

Related U.S. Application Data
(60) Provisional application No. 62/623,982, filed on Jan. 30, 2018.
(60) Provisional application No. 62/586,711, filed on Nov. 15, 2017.

(51) Int. Cl. A61B 5/00; A61B 1/07; A61B 1/04; A61B 1/06; A61B 1/00; A61B 10/02

(52) U.S. Cl. CPC A61B 5/0091; A61B 1/07; A61B 1/043; A61B 10/0233; A61B 1/00117; A61B 5/0071; A61B 1/0638

(57) ABSTRACT

In accordance with aspects of the present disclosure, an exemplary system includes a hollow probe having a lumen containing one or more excitation optical fiber(s) and one or more imaging optical fiber(s) where the probe is sized to access a person's body, a first light source optically coupled to the excitation fiber(s) and configured to emit light that excites fluorescence of NADH in breast tissue, a second light source optically coupled to the excitation fiber(s) and configured to emit light that excites fluorescence of FAD in tissue such as breast tissue, an image capturing device optically coupled to the imaging fiber(s), and a controller configured to control the first light source and the image capturing device to capture NADH fluorescence signals/intensities while the probe is within the person's body and control the second light source and the image capturing device to capture FAD fluorescence signals/intensities while the probe is within the person's body.

### CROSS-REFERENCE TO RELATED APPLICATION

This application claims the benefit of, and priority to, U.S. Provisional Patent Application No. 62/586,711, entitled “REDOX IMAGING BIOPSY NEEDLE FOR BREAST CANCER DIAGNOSIS,” filed on Nov. 15, 2017, and U.S. Provisional Patent Application No. 62/623,982, entitled “REDOX IMAGING BIOPSY NEEDLE FOR BREAST CANCER DIAGNOSIS,” filed on Jan. 30, 2018. The entire contents of each of the foregoing applications are hereby incorporated by reference.

### GOVERNMENT LICENSE RIGHTS

This invention was made with government support under R01CA191207 awarded by National Institutes of Health. The government has certain rights in the invention.

### BACKGROUND

### Technical Field

The present disclosure relates generally to redox ratio and, more particularly, to optical imaging to determine redox ratio.

### Related Art

It is now common knowledge that cancerous cells grow and spread throughout the body after initially manifesting. This process whereby cancer cells break away from the original tumor and travel through the blood or lymph system in order to form new tumors in other organs or tissues of the body is known as metastasis.

Endogenous substances are those that originate from within an organism, tissue, or cell, and fluorophores are fluorescent chemical compounds that can re-emit light upon light excitation. Most notably, fluorophores are used to stain tissues, cells, or materials in analytical methods like fluorescent imaging and spectroscopy. Therefore, endogenous tissue fluorophores that are readily available within the human body provide a fast and inexpensive method for assessing the extent to which cancerous tumors in the body have metastasized. More specifically, endogenous tissue fluorophores allow doctors to evaluate the metabolic rate of cells through optical imaging. Knowledge of the specific metabolic pathways utilized by breast cancer cells may play an important role in determining their invasive and migratory tendencies.

Metabolism refers to the process by which cells break down food/fuel and convert it into energy. Cancer cells rely on an electron transport chain as their primary mechanism of energy production. The electron transport chain produces energy in the form of Adenosine Triphosphate (ATP) by transferring electrons to molecular oxygen. This transfer of electrons occurs by way of a chemical reaction in which the oxidation states of atoms are changed. These chemical reactions involve two complimentary processes: (i) oxidation, wherein a first atom is stripped of a number of electrons, and (ii) reduction, wherein a second atom obtains a number of electrons.

More specifically, in the context of cancer cells this oxidation-reduction reaction can be measured to gauge the metabolic state of the cell. There are two endogenous fluorophores in human body tissue related to cellular metabolism in the electron transport chain: (i) a reduced form of nicotinamide adenine dinucleotide (NADH), which transfers electrons to molecular oxygen in a process known as oxidative phosphorylation, and (ii) flavin adenine dinucleotide (FAD), which receives additional electrons through a process known as glucose catabolism. Correspondingly, an approximation of the oxidation-reduction reaction of the mitochondrial matrix space can be determined from the “redox ratio”, which is the fluorescence intensity of FAD divided by the fluorescence intensity of NADH,

$$( Redox = \frac{FAD}{NADH} ) ,$$

or which is

$$( Redox = \frac{FAD}{NADH + FAD} ) .$$

Optical imaging of the endogenous fluorescence of NADH and FAD presents a non-destructive and label-free method for assessing cell metabolism, because NADH and FAD are metabolic cofactors that play a critical role in the generation of cellular energy through oxidative phosphorylation. Changes in the redox ratio of a cell can be interpreted as a relative change in the rate of glucose catabolism to oxidative phosphorylation. During oxidative phosphorylation, NADH fluorescence decreases due to conversion to non-fluorescent NAD+, and FAD fluorescence increases due to its generation from non-fluorescent FADH2, leading to an increase in the redox ratio. The absence of oxygen or a need to increase glucose catabolism leads to a build-up of NADH that does not get converted to NAD+, causing an increase in NADH fluorescence and a decrease in the redox ratio.

This optical redox ratio can provide the relative changes in the oxidation-reduction state in the cell without the use of exogenous stains or dyes. This advantage is important because it eliminates possible artifacts in metabolic measurements that can be introduced by tissue excision, processing or staining. Accordingly, there is continuing interest in developing and improving optical imaging of redox ratio to track metabolic changes during cell differentiation and malignant transformation.

### SUMMARY

The present disclosure relates to systems and methods for optical imaging to determine redox ratio using a probe containing optical fibers while the probe is within the body of a person. When the probe is used with a needle for accessing the body, such systems and methods may be referred to herein as “needle redox imaging.”

In accordance with aspects of the present disclosure, a system includes a hollow probe having a lumen containing at least one excitation optical fiber and at least one imaging optical fiber where the hollow probe is sized to access the body of a person, a first light source optically coupled to the at least one excitation optical fiber where the first light source is configured to emit light that excites fluorescence of nicotinamide adenine dinucleotide (NADH) in breast tissue, a second light source optically coupled to the at least one excitation optical fiber where the second light source is configured to emit light that excites fluorescence of flavin adenine dinucleotide (FAD) in breast tissue, an image capturing device optically coupled to the at least one imaging optical fiber, and a controller coupled to the first light source, the second light source, and the image capturing device. The controller is configured to control the first light source and the image capturing device to capture NADH fluorescence data while the hollow probe is within the body of the person and configured to control the second light source and the image capturing device to capture FAD fluorescence data while the hollow probe is within the body of the person.

In various embodiments, the first light source is configured to emit 375 nm light, and the second light source is configured to emit 473 nm light.

In various embodiments, the system further includes a first optical element optically coupled to the first light source, the second light source, and the at least one excitation optical fiber, where the first optical element is configured to optically couple both light from the first light source and light from the second light source to the at least one excitation optical fiber. In various embodiments, the first optical element is a dichroic short pass mirror that is angled forty-five degrees relative to the light from the first light source and relative to the light from the second light source.

In various embodiments, the at least one excitation optical fiber includes a plurality of excitation optical fibers, where the plurality of excitation optical fibers entirely surrounds all of the at least one imaging optical fiber at a distal portion of the hollow probe.

In various embodiments, the at least one imaging optical fiber includes a fiber bundle having a substantially circular cross-section at the distal portion of the hollow probe.

In various embodiments, the controller is further configured to diagnose breast cancer based on heterogeneity of the NADH fluorescence data across the substantially circular cross-section and heterogeneity of the FAD fluorescence data across the substantially circular cross-section.

In various embodiments, the controller is configured to diagnose breast cancer based on the NADH fluorescence data and the FAD fluorescence data while the hollow probe is within the body of the person.

In various embodiments, the image capturing device utilizes an exposure time that does not saturate the NADH fluorescence data or the FAD fluorescence data over a measurement range of interest.

In various embodiments, the system further includes a biopsy needle sized to hold the hollow probe within the biopsy needle.

In accordance with aspects of the present disclosure, a method includes receiving an indication that a hollow probe has been inserted into the body of a person where the hollow probe has a lumen containing at least one excitation optical fiber and at least one imaging optical fiber, activating a first light source optically coupled to the at least one excitation optical fiber where the first light source is configured to emit light that excites fluorescence of nicotinamide adenine dinucleotide (NADH) in breast tissue, activating a second light source optically coupled to the at least one excitation optical fiber where the second light source is configured to emit light that excites fluorescence of flavin adenine dinucleotide (FAD) in breast tissue, conveying the NADH fluorescence and the FAD fluorescence in the at least one imaging optical fiber, capturing, by an image capturing device optically coupled to the at least one imaging optical fiber, image data based on the NADH fluorescence and the FAD fluorescence conveyed in at least one imaging optical fiber, controlling the first light source and the image capturing device to capture the image data based on the NADH fluorescence while the hollow probe is within the body of the person, and controlling the second light source and the image capturing device to capture the image data based on the FAD fluorescence while the hollow probe is within the body of the person.

In various embodiments, the first light source is configured to emit 375 nm light, and the second light source is configured to emit 473 nm light.

In various embodiments, the method further includes optically coupling, by a first optical element, both light from the first light source and light from the second light source to the at least one excitation optical fiber. In various embodiments, the first optical element is a dichroic short pass mirror that is angled forty-five degrees relative to the light from the first light source and relative to the light from the second light source.

In various embodiments, the at least one excitation optical fiber includes a plurality of excitation optical fibers, where the plurality of excitation optical fibers entirely surrounds all of the at least one imaging optical fiber at a distal portion of the hollow probe.

In various embodiments, the at least one imaging optical fiber includes a fiber bundle having a substantially circular cross-section at the distal portion of the hollow probe.

In various embodiments, the method further includes diagnosing breast cancer based on heterogeneity of the NADH fluorescence data across the substantially circular cross-section and heterogeneity of the FAD fluorescence data across the substantially circular cross-section.

In various embodiments, method further includes diagnosing breast cancer based on the NADH fluorescence and the FAD fluorescence while the hollow probe is within the body of the person.

In various embodiments, the method further includes calibrating an exposure time of the image capturing device that does not saturate the NADH fluorescence data or the FAD fluorescence data over a measurement range of interest.

In various embodiments, controlling the first light source and controlling the second light source includes alternating the first light source and the second light source ON and OFF such that the first light source and the second light source are not simultaneously ON.

Further details and aspects of exemplary embodiments of the present disclosure are described in more detail below with reference to the appended figures.

### BRIEF DESCRIPTION OF THE DRAWINGS

The accompanying drawings, which are incorporated in and constitute a part of the specification, illustrate embodiments of the disclosure and together with a general description of the disclosure given above, and the detailed description of the embodiment(s) given below, serve to explain the principles of the present disclosure.

FIG. 1 is a diagram of an exemplary system for optical redox imaging;

FIG. 2 is a diagram of an exemplary optical fiber configuration within a distal portion of the probe of FIG. 1;

FIG. 3 is a diagram of exemplary phantoms used for system characterization and/or testing;

FIG. 4 is a plot of exemplary NADH measurement information used for characterizing system sensitivity;

FIG. 5 is a plot of exemplary FAD measurement information used for characterizing system sensitivity;

FIG. 6 is a plot of exemplary NADH measurement information used for characterizing depth-dependent signal profile;

FIG. 7 is a plot of exemplary FAD measurement information used for characterizing depth-dependent signal profile;

FIG. 8 is an exemplary plot for determining NADH measurement correlation between the disclosed system and the Chance redox scanner;

FIG. 9 is an exemplary plot for determining FAD measurement correlation between the disclosed system and the Chance redox scanner;

FIG. 10 is a diagram of exemplary optical imaging using the disclosed system before and after introducing rotenone;

FIG. 11 is a diagram of exemplary optical imaging using the disclosed system before and after introducing FCCP;

FIG. 12 is a diagram of exemplary measurement values before and after introducing rotenone; and

FIG. 13 is a diagram of exemplary measurement values before and after introducing FCCP.

### DETAILED DESCRIPTION

The present disclosure relates to systems and methods for optical imaging to determine redox ratio using a probe containing optical fibers while the probe is within the body of a person. When the probe is used with a needle for accessing the body, such systems and methods may be referred to herein as “needle redox imaging.” Aspects of optical redox imaging are described in U. Kanniyappan et al., “Novel needle redox endoscopy imager for cancer diagnosis,” Proc. SPIE 10489, Optical Biopsy XVI: Toward Real-Time Spectroscopic Imaging and Diagnosis, 104890J (26 Feb. 2018), which is hereby incorporated by reference in its entirety. Aspects and embodiments are described in detail with reference to the drawings, in which like or corresponding reference numerals designate identical or corresponding elements in each of the several views.

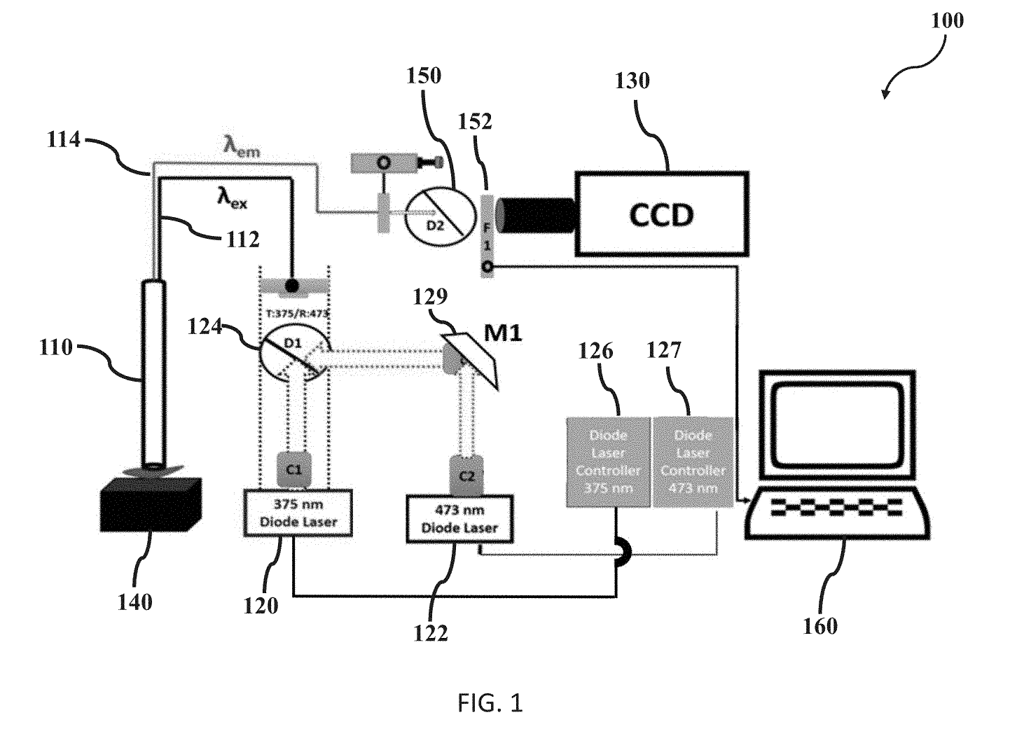

FIG. 1 is a diagram of an exemplary system 100 for optical redox imaging. The illustrated system includes a probe 110 containing optical fibers 112, 114, a light source 120 that emits light to excite NADH fluorescence, a light source 122 that emits light to excite FAD fluorescence, an image capturing device 130, and a controller (not shown). The system also includes various other components, such as optical elements and control wiring, among other things.

In accordance with aspects of the present disclosure, the probe 110 is sized and dimensioned to enter the body of a person and to access body tissue such as breast tissue. In various embodiments, the probe 110 can be a hollow needle or have the shape of a hollow needle. In various embodiments, the probe can be separate from a biopsy needle, such that the probe can have an outer diameter of approximately 2.706 mm and can fit inside a 11G biopsy needle.

The probe 110 contains one or more excitation optical fibers 112 that are optically coupled to the light sources. As used herein, “optical coupling” or “optically coupled” refers to being connected by a path of directed light. Accordingly, two components can be optically coupled when there is a path of directed light from one component to the other component. In various embodiments, the optically coupling can be achieved by one or more optical elements 124 that are configured and positioned to direct light from the light sources to the excitation optical fiber(s). For ease of description herein, the term “optically coupled” may be shortened to “coupled,” and “optical coupling” may be shortened to “coupling.”

In the illustrated embodiment, a light source 120 is configured to emit light that excites NADH fluorescence, and this light source 120 may be referred to as “NADH light source.” Another light source 122 is configured to emit light that excites FAD fluorescence, and this light source 122 may be referred to herein as “FAD light source.” In the illustrated embodiment, the NADH light source 120 includes a laser diode that emits 375 nm light and collimation optics, and the FAD light 122 source includes a laser diode that emits 473 nm light and collimation optics. Each light source 120, 122 is connected to a laser diode current controller 126, 127. In various embodiments, the wavelength or wavelengths of light emitted by the light sources 120, 122 may differ from the specific wavelengths illustrated in FIG. 1, as long as they excite NADH and FAD fluorescence.

Both light from the NADH light source 120 and light from the FAD light source 122 are directed to an optical element 124 that directs both light sources to the excitation optical fiber(s) 112. In FIG. 1, light from the NADH light source 120 travels directly to the optical element 124, while light from the FAD light source 122 is redirected by a mirror or optical element 129. The illustrated embodiment is exemplary, and in various embodiments, the light from either or both light sources 120, 122 can travel directly to the optical element 124 or can be redirected by one or more mirrors or optical elements. Such variations are contemplated to be within the scope of the present disclosure.

The optical element 124 which couples light from both light sources 120, 122 to the excitation optical fiber(s) 112 may be a dichroic short pass mirror, such as one from Chroma Technology Corporation. In the illustrated embodiment, the dichroic mirror 124 is configured and positioned to transmit the 375 nm light through to the excitation optical fiber(s) 112 and to reflect/redirect the 473 nm light to the excitation optical fiber(s) 112. In various embodiments, the dichroic mirror 124 is placed at a forty-five degree angle with respect to the optical axis of 473 nm light. In various embodiments, other positioning of the optical element 124 and/or other types of optical elements are contemplated to be within the scope of the present disclosure. For example, the optical element 124 can include one mirror that redirects light from the NADH light source and another mirror that redirects light from the FAD light source. Other variations are contemplated.

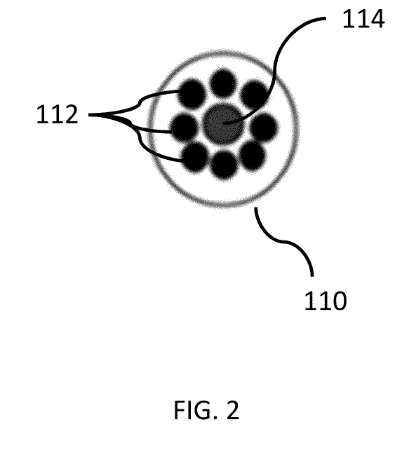

Control of the light sources will be described in more detail later herein. For now, it is sufficient to note that the NADH light source 120 and the FAD light source 122 alternate being ON and OFF, and are not simultaneously ON at the same time. Thus, fluorescence of NADH and FAD are excited by switching ON/OFF between the two light sources 120, 122. The fluorescence of NADH and FAD are picked up by one or more imaging optical fibers 114. The excitation optical fiber(s) 112 and the imaging optical fiber(s) 114 will now be described in connection with FIG. 2.

FIG. 2 is a diagram of an exemplary optical fiber configuration within a distal portion of the probe 110 of FIG. 1, including excitation optical fiber(s) 112 and imaging optical fiber(s) 114. In the illustrated embodiment, the probe 110 contains a fiber bundle 114 at the center, which is surrounded by excitation fibers 112. The illustration is not drawn to scale. In various embodiments, each excitation fiber 112 has a diameter of 400 μm, has a polyimide coating, and has a numerical aperture of 0.22. In various embodiments, the fiber bundle 114 includes 13,500 individual fibers that are each 8.2 μm in diameter. The fiber bundle 114 is arranged to have approximately a circular cross-section with a diameter of about 1 mm. The cross-section is not perfectly circular due to being formed by individual fibers. In various embodiments, the parameters of individual fibers and of the fiber bundle can be different than as described above, including different dimensions, shapes, and/or optical parameters. In various embodiments, the arrangement of fibers and fiber bundles can be different than as illustrated in FIG. 2. For example, the imaging optical fiber bundle 114 may have a cross-section that is another shape. In certain variations, the excitation optical fibers 112 may be positioned in a different arrangement than as shown in FIG. 2. Such variations are contemplated to be within the scope of the present disclosure.

Referring again to FIG. 1, the probe 110 is inserted into the body of a person 140, such as into breast tissue. While the probe 110 is within the body 140, fluorescence of NADH and FAD are excited by switching ON/OFF between the two light sources 120, 122, as described above. After excitation of NADH or FAD, the corresponding fluorescence emission is received and conveyed by the imaging fiber bundle 114.

As persons skilled in the art will understand, in various situations, NADH fluorescence can have wavelengths of about 410 nm-450 nm, and FAD fluorescence can have wavelengths of about 495 nm-535 nm. The imaging fiber bundle 114 can convey these fluorescence emissions to an optical element 150. In various embodiments, the optical element 150 can be a poly-dichroic mirror that transmits 430±20 nm and 515±20 nm through the optical element 150, such as one from Chroma Technology Corporation. In various embodiments, the poly-dichroic mirror 150 can be placed at forty-five degrees with respect to the path of fluorescence emissions exiting the imaging fiber bundle 114, such that the fluorescence emissions are passed through but other light can be reflected. In various embodiments, the optical element 150 can be another type of optical element.

The optical element 150 passes the fluorescence emissions to optical filters 152. In various embodiments, the optical filters 152 can be implemented by motorized rotating filter wheel that includes one band-pass filter for filtering 469±35 nm for NADH emissions, and another band-pass filter for filtering 520±35 nm for FAD emissions. The motorized wheel can be controlled to rotate to the correct filter at the proper timing. The filter wheel 152 is exemplary, and other types and/or numbers of filters are contemplated to be within the scope of the present disclosure.

The filtered emissions are then captured by the image capturing device 130. In various embodiments, the image capturing device 130 can be a cooled, charge-coupled device (CCD). In various embodiments, another type of image capturing device can be used. In accordance with aspects of the present disclosure, the image captured by the image capturing device 130 will have substantially the same shape as the cross-sectional shape of the imaging fiber bundle 114. Additionally, the images are captured while the probe 110 is within the body 140 of the person, such as within breast tissue.

In accordance with aspects of the present disclosure, the images of NADH fluorescence and FAD fluorescence captured by the image capturing device 130 can be communicated to a processor and/or storage, for computation of redox ratio and determination of a cancer diagnosis, such as breast cancer diagnosis. Persons skilled in the art will understand the techniques and computations for doing so, including the techniques and computations discussed in H. N. Xu et al., “Quantitative Mitochondrial Redox Imaging of Breast Cancer Metastatic Potential,” Journal of Biomedical Optics, Vol. 15(3), pp. 036010-1-036010-10, May/June 2010; H. N. Xu et al., “Imaging the Redox States of Human Breast Cancer Core Biopsies,” Adv. Exp. Med. Biol., 765:343-349, 2013; and H. N. Xu et al., “Optical Redox Imaging Indices Discriminate Human Breast Cancer From Normal Tissues,” Journal of Biomedical Optics, Vol. 21(11), pp. 114003-1-114003-8, November 2016, each of which is hereby incorporated by reference in its entirety. For example, magnitude of the redox ratio can be indicative of cancerous tissue, and redox heterogeneity can be indicative of metastatic risk, and these measures can be used to diagnosis breast cancer. In accordance with aspects of the present disclosure, the redox ratio and/or the diagnosis can be determined while the probe 110 is within the body 140 of the person, such as within breast tissue, such that these results can be available to a clinician in real-time. In various embodiments, the diagnosis may not be determined while the probe 110 is within the body of the person 140, but can be determine within the amount of time of a clinical visit, such that these results can be available to a clinician in the same visit that the procedure is performed. The determination and diagnosis can be performed by a computing device 160, such as a desktop, a laptop, server, a tablet, or another type of computing device.

Accordingly, described above are systems and methods for optical imaging to determine redox ratio using a probe containing optical fibers while the probe is within the body of a person. The following describes various aspects of controlling, testing, and/or calibrating the disclosed systems. The following describes the probe as fitting into a needle, such that a needle optical imaging procedure is performed. However, the following disclosure also applies to probes that are not used with needles.

In accordance with aspects of the present disclosure, in calibrating, controlling, and/or testing the disclosed needle redox imaging (“NRI”) system, various parameters and characteristics can be determined. As persons skilled in the art will understand, optical phantoms are tissue-simulating objects used to mimic light propagation in living tissue. Phantoms can be used for characterizing the NRI system.

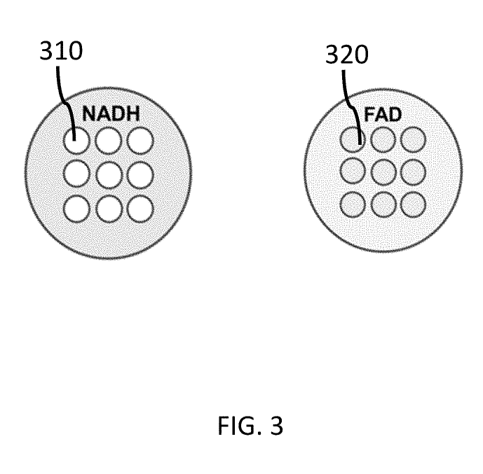

In accordance with aspects of the present disclosure, the sensitivity of the needle redox imager (“NM”) can be quantified. Two liquid phantom matrices, one for NADH and one for FAD, can be prepared. In various embodiments, the liquid includes phosphate-buffered saline, 20% intralipid, and NADH or FAD. Prior to NRI characterization, the intralipid concentration can be optimized to 3.3% v/v to produce the reduced scattering coefficient 18 cm⁻¹ for NADH and 16 cm⁻¹ for FAD, as seen in breast tissue. In various embodiments, nine or ten different concentrations of NADH and FAD can be prepared, including some or all of 0.97, 1.95, 7.81, 15.62, 31.25, 62.5, 125, 150, 500, and/or 1000 μM. The prepared liquid can be filled into a black well plate to carry out measurements, as shown in FIG. 3. In various embodiments, other liquid formulations can be used to represent different types of tissue, and different concentrations can be used for different measurement ranges of interest.

With reference to FIG. 1 and FIG. 3, the two liquid phantom matrices 310, 320 can be used to quantify the sensitivity limit of the NRI. To perform the sensitivity characterization, the tip of the NRI needle 110 is positioned inside the liquid phantom, and images are obtained by the image capturing device 130 at various exposure times, such as 100 ms, 500 ms, and 1000 ms exposure times. In various embodiments, the duration and number of exposure times can vary. All images can be corrected for non-uniform illumination using flat-field images, which can be acquired from uniform turbid epoxy resin fluorescence phantoms for FAD and NADH. The image processing can be performed by the computing device 160.

In various embodiments, the observed sample images are divided by the reference phantom images and multiplied by the averaged intensity of the reference phantom. In various embodiments, the limit of detection can be estimated by the protocol approved by International Council for Harmonisation (ICH), Q2(R1)—Validation of analytical procedures: Text And Methodology. In such manner, the limit of detection (LOD) can be calculated using the following formula:

$$LOD = \frac{3.3 σ}{S} ,$$

where σ is the standard deviation of the background (the blank), and S is the slope of the calibration curve in the linear range. Additionally, the limit of quantification (LOQ) is estimated using the following formula:

$$LOQ = \frac{10 σ}{S} .$$

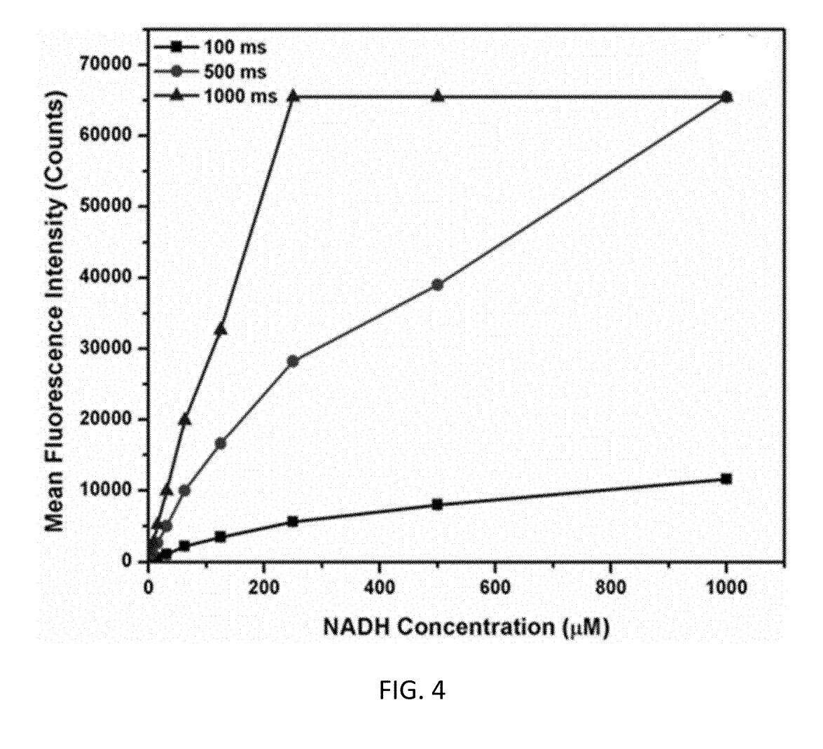

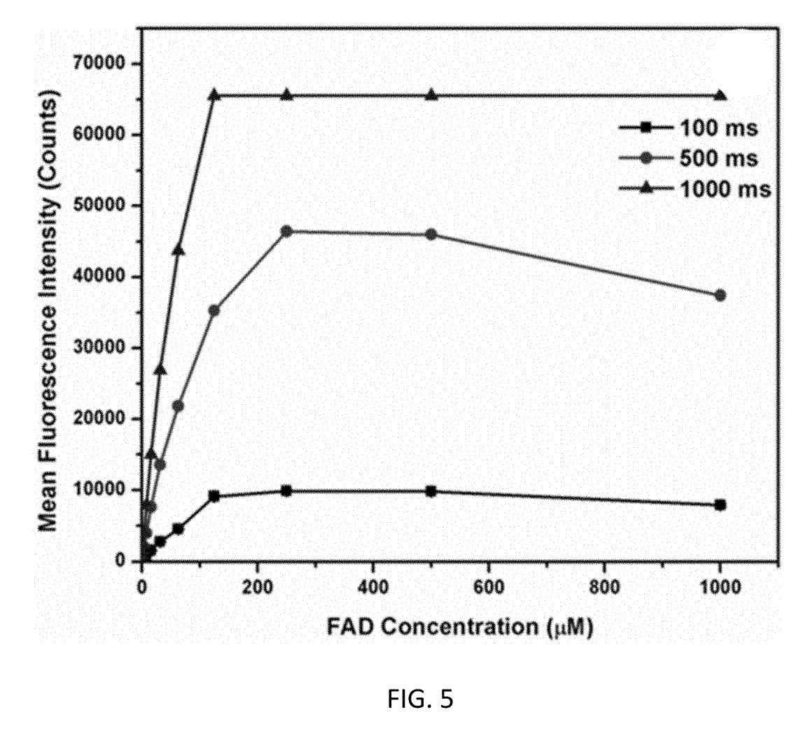

With respect to the slope of the calibration curve in the linear region (S), the parameter can be determined based on plotting varying concentration vs. mean fluorescence intensity, as shown in the examples of FIG. 4 and FIG. 5. The region of interest for computing the mean fluorescence intensity can be 200×200 pixels. In various embodiments, another region of interest size can be used. All images are corrected by the flat-field correction technique with background subtracted. The image processing and computations can be performed by the computing device 160.

In FIG. 4, at lower concentration (<10 μM), NADH phantom exhibits minimal/insignificant difference in intensity. As NADH concentration increases the fluorescence intensity starts to increase linearly until 250 μM, after which the fluorescence reaches saturation with 1000 ms exposure time. In FIG. 5, on the other hand, FAD fluorescence intensity increases up to 125 μM and then reaches a plateau for all exposure times due to fluorescence quenching. Using the plots in FIG. 4 and FIG. 5, the slope of the linear portion can be determined as the parameter S.

Using the techniques described above, the sensitivity of the NRI can be characterized, and an example is provided for illustration in Table 1, in which concentration sensitivity is displayed molars (M). In various embodiments, other ways of determining sensitivity can be used, and such variations are contemplated to be within the scope of the present disclosure.

In various embodiments, the results of sensitivity characterization can be used to control or calibrate the NRI system. For example, in various embodiments, the plots in FIG. 4 and FIG. 5 can be used to set an exposure time of the image capturing device such that the fluorescence data being captured will not saturate, and such that the limits of detection and quantification are sufficient for the measurement of interest.

TABLE 1

| | Exposure | Limit of | Limit of |
|---|---|---|---|
| | Time | Detection | Quantification |
| Fluorophore | (ms) | (μM) | (μM) |
| NADH | 100 | 3.2 | 9.75 |
| | 500 | 1.5 | 4.52 |
| | 1000 | 0.98 | 3.0 |
| FAD | 100 | 1.11 | 3.37 |
| | 500 | 0.28 | 0.9 |
| | 1000 | 0.20 | 0.60 |

In accordance with aspects of the present disclosure, the resolution of the needle redox imager (“NRI”) can be quantified. In various embodiments, the resolution characterization can apply the ISO endoscope standard, which recommends the use of a resolution target (e.g., the USAF 1951 target) to visually identify resolution in horizontal and vertical directions at the center. In various embodiments, the resolution characterization can use a version of the standard bar chart approach in which a negative target (e.g., Positive 1951 USAF Test Target) is trans-illuminated by white light and images are recorded by the image capturing device 130 after light passes through the filter wheel. In various embodiments, other targets can be used. The contrast transfer function can be calculated using the formula:

$${C}_{I} = \frac{( {I}_{max} - {I}_{min} )}{( {I}_{max} + {I}_{min} )} ,$$

where Iₘₐₓ is the maximum intensity at the bright region, and Iₘᵢₙ is the minimum intensity at the dark region. Then, using the contrast transfer function, the spatial resolution can be calculated using Rayleigh criterion defined as the value corresponding to a contrast value of 26.4%, as described in Lasch, P. and Naumann, D., “Spatial resolution in infrared microspectroscopic imaging of tissues,” BBA Biomembrane 1758(7), 814-829 (2006). Based on these techniques, the spatial resolution of the NRI system can be characterized, and an example is provided for illustration in Table 2.

TABLE 2

| | Spatial |
|---|---|
| | Resolution |
| Fluorophore | (μm) |
| NADH | 111 |
| FAD | 88 |

The image processing and computations can be performed by the computing device 160. In various embodiments, the result of the spatial resolution characterization can be used for aspects of the NRI system that rely on spatial information. In various embodiments, other ways of determining spatial resolution can be used, and such variations are contemplated to be within the scope of the present disclosure.

In accordance with aspects of the present disclosure, the depth-dependent signal profiles of NADH and FAD can be quantified. In various embodiments, to determine the depth-dependent signal profiles of NADH and FAD, an epoxy resin solid phantom can be prepared. The phantom can include a resin and hardener ratio of 1:1, which can be mixed with NADH or FAD solutions of 100 μM to generate the phantom. In various embodiments, the thickness of the flat solid phantom is about 3 mm. For each depth measurement, a phosphate-buffered saline (PBS) with intralipid (3.3% v/v) solution can be added to the solid phantom, and the NRI probe can be positioned touching the top surface of the solution. The blank (intralipid+PBS) solution can be slowly added to increase the height of the NRI probe from the fluorescent solid phantom to study the depth dependence of fluorescence intensity. In various embodiments, the height of the blank solution from the solid phantom surface can be determined using optical coherence tomography (OCT).

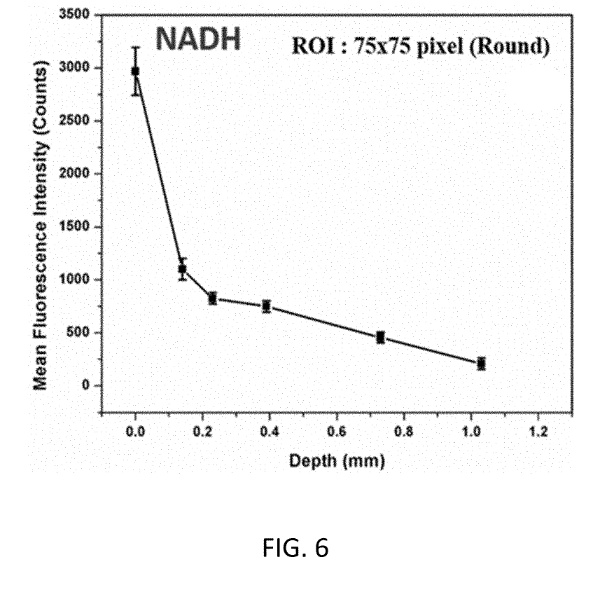

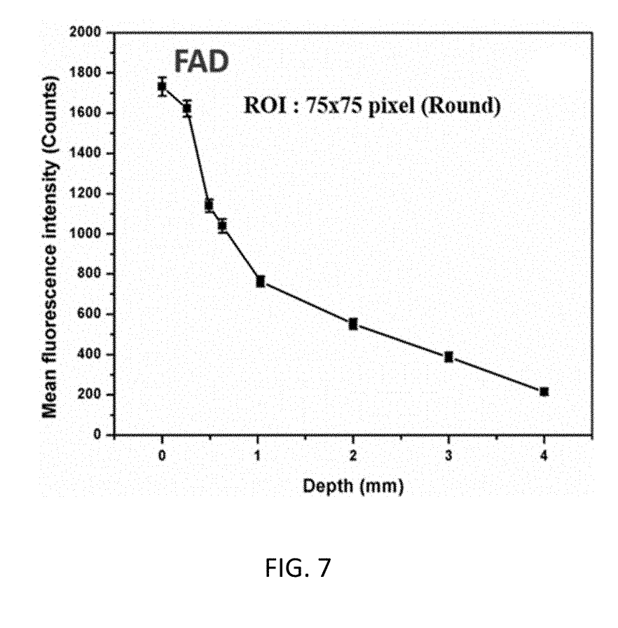

Using these techniques, depth-dependent signal profiles of NADH and FAD can be quantified, and examples are shown in FIGS. 6 and 7. The image processing and computations can be performed by the computing device 160. In FIG. 6, the NADH plot shows steep decrease in intensity with increase in depth to ˜0.1 mm. In FIG. 7, the FAD phantom exhibits slower decrease in intensity with respect to depth. Based on the characterization, the signal detection depth range is approximately 1 mm for NADH and approximately 4 mm for FAD.

Based on characterizing the depth-dependent signal profiles, the NRI system can provide information regarding what is being imaged in the body of the patient relative to the position of the probe needle, and such information can be used in improving redox ratio determinations.

In accordance with aspects of the present disclosure, performance and/or accuracy of the NRI system can be compared with the Chance redox scanner, which persons skilled in the art will recognize. For comparing NRI to the Chance redox scanner, NADH and FAD phantom matrices can be prepared with concentrations of 1.95, 3.90, 7.81, 15.62, 31.25, 15.63, 31.25, 62.5, 125, and 250 μM. A phosphate-buffered saline with intralipid (3.3% v/v) can be used for serial dilution. The phantom matrices can be snap-frozen and milled flat before scanning/imaging by both the Chance redox scanner and NRI probe. Similar to the operation of the Chance redox scanner, the NRI probe can be positioned 80 μm above the phantoms surface.

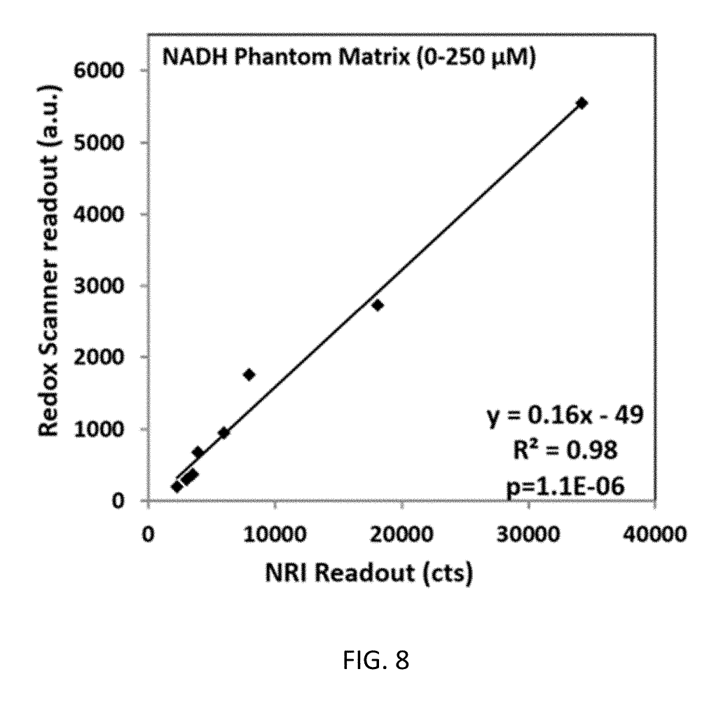

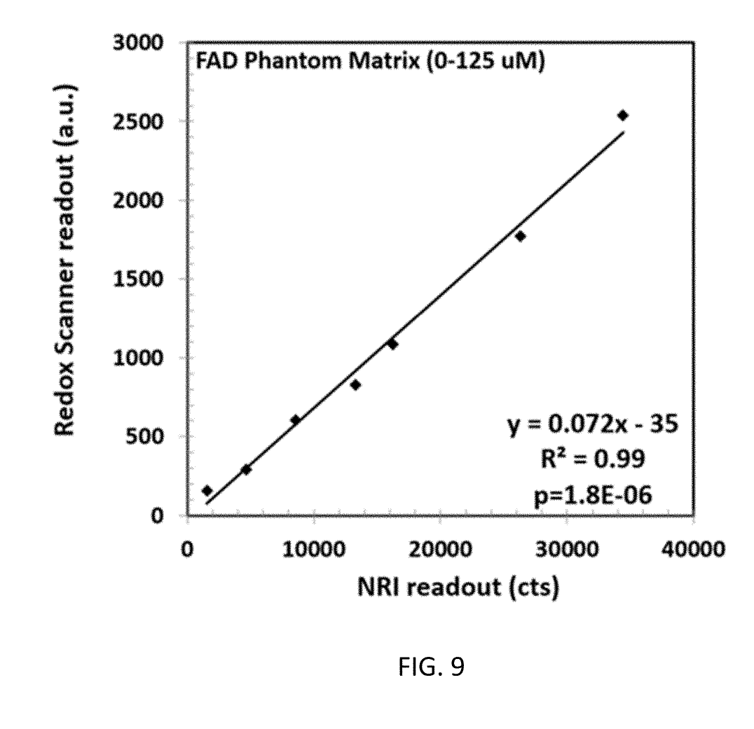

Exemplary detection results are shown in FIGS. 8 and 9. In FIG. 8, linear regression analysis of the NADH phantom matrix shows that both systems exhibit a similar linearity in the range between 0 to 250 μM. In FIG. 9, the FAD phantom matrix for both systems shows a linearity range between 0 to 125 μM. Based on this characterization, there is good linear correlation between the two instruments for NADH ranging from 0-250 μM and FAD ranging from 0-125 μM.

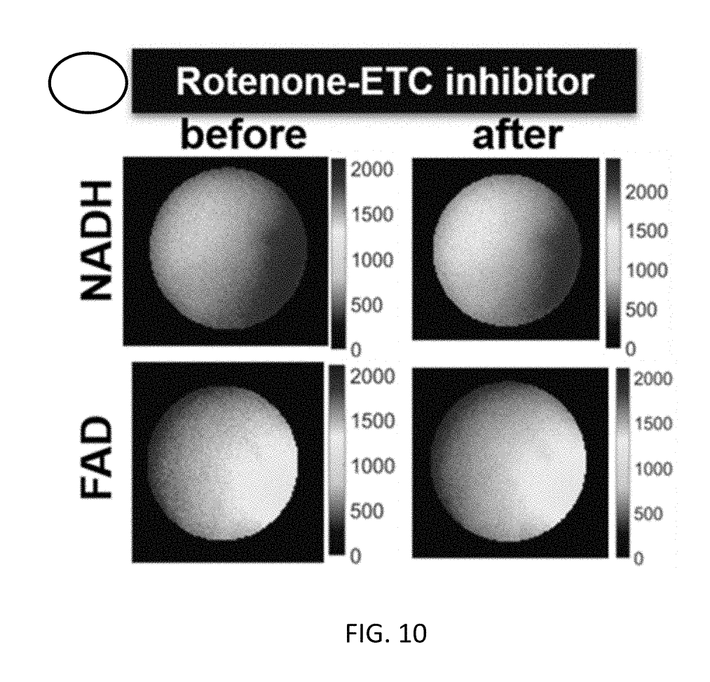

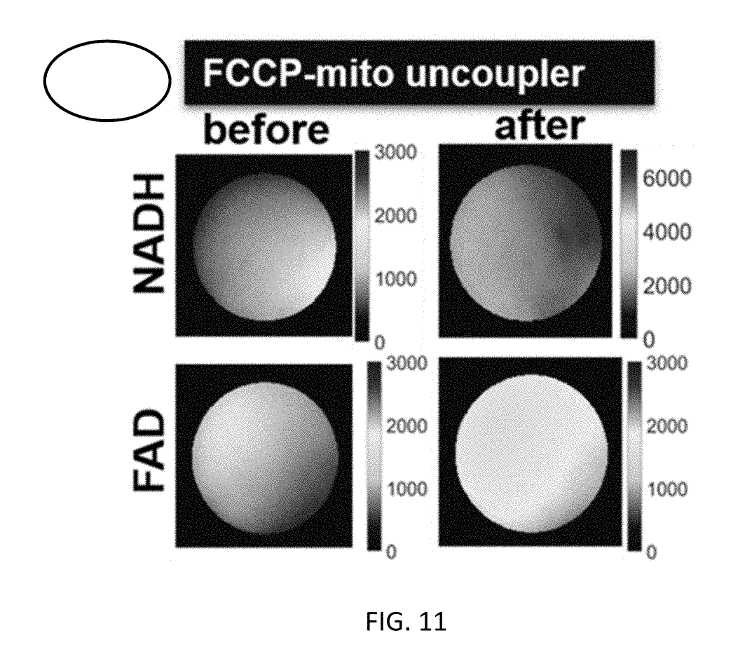

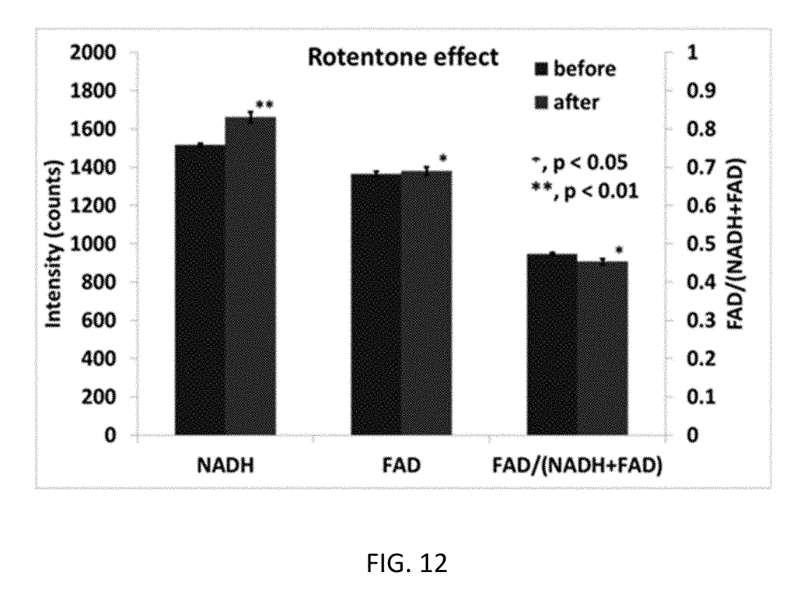

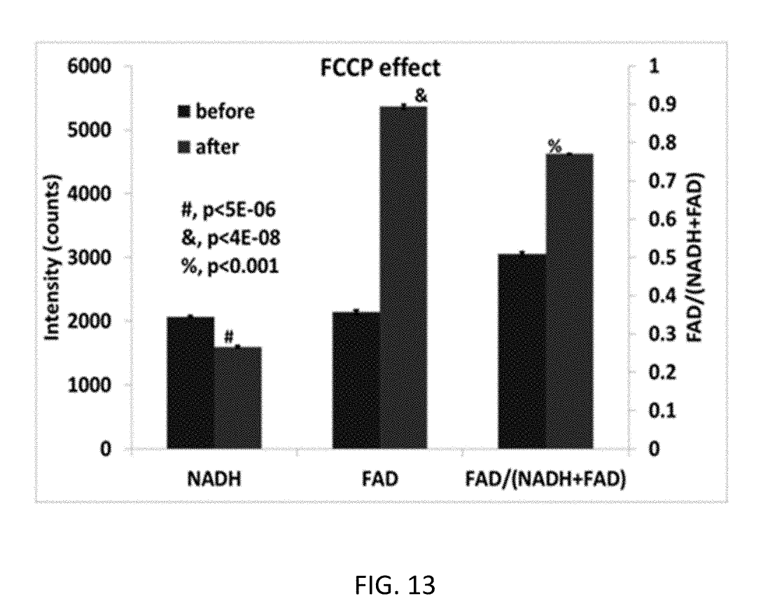

In accordance with aspects of the present disclosure, performance of the NRI system can be tested to determine accuracy using tissue rather than phantom. In various embodiments, a mouse muscle tissue sample can be measured using the NRI. A frozen mice can be thawed to room temperature. Then, a small piece of the muscle tissue can be removed using a surgical scalpel and carefully immersed in saline solution. The removed tissue slices can be immersed in a buffer, and later added with 10 μM of rotenone or carbonylcyanide-p-trifluoromethoxyphenylhydrazone (FCCP) in a petri dish for measurements. The fluorescence images can be recorded before and after adding the drugs, with the background corrected, as shown in FIGS. 10 and 11. The NADH, FAD, and redox ratios can be averaged across multiple locations in the images to obtain their mean values, as shown in FIGS. 12 and 13. In various embodiments, standard deviations and t-test can be used to determine the statistical significance of the differences induced by the treatments. The image processing and computations can be performed by the computing device 160. The mean values in FIGS. 12 and 13 can be used to determine that the NRI system detects expected changes in NADH, FAD, and redox ratio, as expected when retontone and FCCP are introduced.

Accordingly, described above are systems and methods for characterizing, calibrating, and/or testing the NRI system. The following will describe controls for the system of FIG. 1. Referring again to FIG. 1, the system can be controlled by a controller, which can be a computing device 160 or can be a standalone device (not shown) that is separate from a computing device 160. For the purpose of this description and for ease of explanation, the computing device 160 will be referred to as the controller. The controller 160 can include instructions performed by a processor and/or by hardware circuitry, and can include communication interface to various components of the system 100, such a signal wires. The instructions, when executed by the processor, can cause signals to be conveyed to various components, or hardware circuitry can do so. Functions of the controller 160 can include timing, such as controlling the NADH light source 120 and the FAD light source 122 to alternate ON and OFF, controlling the filter wheel 152 to place the correct filter at the correct time, and controlling the image capturing device 130 to capture the fluorescent emissions at a specified exposure time and at the correct timing, to effectuate the operations described above herein. In various embodiments, the controller/computing device 160 can also perform any of the image processing and computations described herein. In various embodiments, the controller may not perform any image processing or computations, and rather, the computing device 160 may do so.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, causes the one or more processors to perform one or more methods and/or algorithms.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

1. A system comprising:
a hollow probe having a lumen containing at least one excitation optical fiber and at least one imaging optical fiber, the hollow probe sized to access the body of a person;
a first light source optically coupled to the at least one excitation optical fiber, the first light source configured to emit light that excites fluorescence of nicotinamide adenine dinucleotide (NADH) in breast tissue;
a second light source optically coupled to the at least one excitation optical fiber, the second light source configured to emit light that excites fluorescence of flavin adenine dinucleotide (FAD) in breast tissue;
an image capturing device optically coupled to the at least one imaging optical fiber; and
a controller coupled to the first light source, the second light source, and the image capturing device, the controller configured to control the first light source and the image capturing device to capture NADH fluorescence data while the hollow probe is within the body of the person and configured to control the second light source and the image capturing device to capture FAD fluorescence data while the hollow probe is within the body of the person.

2. The system of claim 1, wherein the first light source is configured to emit 375 nm light, and wherein the second light source is configured to emit 473 nm light.

3. The system of claim 1, further comprising a first optical element optically coupled to the first light source, the second light source, and the at least one excitation optical fiber, wherein the first optical element is configured to optically couple both light from the first light source and light from the second light source to the at least one excitation optical fiber.

4. The system of claim 3, wherein the first optical element is a dichroic short pass mirror that is angled forty-five degrees relative to the light from the first light source and relative to the light from the second light source.

5. The system of claim 1, wherein the at least one excitation optical fiber includes a plurality of excitation optical fibers, wherein the plurality of excitation optical fibers entirely surrounds all of the at least one imaging optical fiber at a distal portion of the hollow probe.

6. The system of claim 1, wherein the at least one imaging optical fiber includes a fiber bundle having a substantially circular cross-section at the distal portion of the hollow probe.

7. The system of claim 6, wherein the controller is further configured to diagnose breast cancer based on heterogeneity of the NADH fluorescence data across the substantially circular cross-section and heterogeneity of the FAD fluorescence data across the substantially circular cross-section and heterogeneity of the redox ratio across the substantially circular cross-section.

8. The system of claim 1, wherein the controller is further configured to diagnose breast cancer based on the NADH fluorescence data and the FAD fluorescence data while the hollow probe is within the body of the person.

9. The system of claim 1, wherein the image capturing device utilizes an exposure time that does not saturate the NADH fluorescence data or the FAD fluorescence data over a measurement range of interest.

10. The system of claim 1, wherein further comprising a biopsy needle sized to hold the hollow probe within the biopsy needle.

11. A method comprising:
receiving an indication that a hollow probe has been inserted into the body of a person, the hollow probe having a lumen containing at least one excitation optical fiber and at least one imaging optical fiber;
activating a first light source optically coupled to the at least one excitation optical fiber, the first light source configured to emit light that excites fluorescence of nicotinamide adenine dinucleotide (NADH) in breast tissue;
activating a second light source optically coupled to the at least one excitation optical fiber, the second light source configured to emit light that excites fluorescence of flavin adenine dinucleotide (FAD) in breast tissue;
conveying the NADH fluorescence and the FAD fluorescence in the at least one imaging optical fiber;
capturing, by an image capturing device optically coupled to the at least one imaging optical fiber, image data based on the NADH fluorescence and the FAD fluorescence conveyed in at least one imaging optical fiber;
controlling the first light source and the image capturing device to capture the image data based on the NADH fluorescence while the hollow probe is within the body of the person; and
controlling the second light source and the image capturing device to capture the image data based on the FAD fluorescence while the hollow probe is within the body of the person.

12. The method of claim 11, wherein the first light source is configured to emit 375 nm light, and wherein the second light source is configured to emit 473 nm light.

13. The method of claim 11, further comprising optically coupling, by a first optical element, both light from the first light source and light from the second light source to the at least one excitation optical fiber.

14. The method of claim 13, wherein the first optical element is a dichroic short pass mirror that is angled forty-five degrees relative to the light from the first light source and relative to the light from the second light source.

15. The method of claim 11, wherein the at least one excitation optical fiber includes a plurality of excitation optical fibers, wherein the plurality of excitation optical fibers entirely surrounds all of the at least one imaging optical fiber at a distal portion of the hollow probe.

16. The method of claim 11, wherein the at least one imaging optical fiber includes a fiber bundle having a substantially circular cross-section at the distal portion of the hollow probe.

17. The method of claim 16, further comprising diagnosing breast cancer based on heterogeneity of the NADH fluorescence data across the substantially circular cross-section and heterogeneity of the FAD fluorescence data across the substantially circular cross-section and heterogeneity of the redox ratio across the substantially circular cross-section.

18. The method of claim 11, further comprising diagnosing breast cancer based on the NADH fluorescence and the FAD fluorescence while the hollow probe is within the body of the person.

19. The method of claim 11, further comprising calibrating an exposure time of the image capturing device that does not saturate the NADH fluorescence intensities or the FAD fluorescence intensities over a measurement range of interest.

20. The method of claim 11, wherein controlling the first light source and controlling the second light source includes alternating the first light source and the second light source ON and OFF such that the first light source and the second light source are not simultaneously ON.
